SEQUENCE LISTING (24 sequences)

SEQ ID NO: 1 - protein, 227 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 2 - protein, 478 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 3 - protein, 488 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRT

SEQ ID NO: 4 - protein, 347 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 5 - protein, 357 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 6 - protein, 608 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 7 - protein, 608 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRT

SEQ ID NO: 8 - protein, 227 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 9 - protein, 478 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 10 - protein, 488 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRT

SEQ ID NO: 11 - protein, 347 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 12 - protein, 357 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 13 - protein, 608 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 14 - protein, 608 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRT

SEQ ID NO: 15 - protein, 10 aa, synthetic construct
GGGGSGGGGS

SEQ ID NO: 16 - protein, 5 aa, synthetic construct
GGGGS

SEQ ID NO: 17 - protein, 251 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRT

SEQ ID NO: 18 - protein, 120 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 19 - protein, 12 aa, synthetic construct
ESKYGPPCPPCP

SEQ ID NO: 20 - protein, 215 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 21 - protein, 215 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 22 - protein, 125 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 23 - protein, 111 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRT

SEQ ID NO: 24 - protein, 15 aa, synthetic construct
GGGGSGGGGSGGGGS

(19) United States
(12) Patent Application Publication
(10) Pub. No.: US 2024/0425590 A1
(43) Pub. Date: Dec. 26, 2024

(54) MULTISPECIFIC ANTIBODIES AND USES THEREOF

(71) Applicant: Vibrant Pharma Limited, Guangzhou (CN)

(72) Inventors: Jianbing Zhang, Guangzhou (CN); Luquan Wang, Guangzhou (CN); Changhua Zhou, Guangzhou (CN)

(21) Appl. No.: 18/698,522

(22) PCT Filed: Nov. 7, 2022
(86) PCT No.: PCT/CN2022/130330

§ 371 Date: Apr. 4, 2024

(30) Foreign Application Priority Data
Nov. 5, 2021 (WO) PCT/CN2021/129062

(51) Int. Cl. C07K 16/28; A61K 47/68; A61P 35/00; C07K 16/30

(52) U.S. Cl. CPC C07K 16/2809; A61K 47/6849; A61K 47/6853; A61P 35/00; C07K 16/3007; C07K 2317/14; C07K 2317/31; C07K 2317/524; C07K 2317/526; C07K 2317/53; C07K 2317/569; C07K 2317/622; C07K 2317/92

(57) ABSTRACT

This disclosure relates to multispecific antibodies (e.g., bispecific antibodies) or antigen-binding fragments thereof. In one aspect, the multispecific antibodies or antigen-binding fragments thereof can bind to a T cell antigen (e.g., CD3) and/or a tumor-associated antigen (e.g., CEACAM5), or a combination thereof.

### TECHNICAL FIELD

This disclosure relates to multispecific antibodies or antigen-binding fragments thereof.

### BACKGROUND

Naturally occurring antibodies typically only target one antigen. A multispecific antibody can be manufactured in different structural formats, so that they can simultaneously bind to two or more different epitopes. These epitopes can be in the same antigen or in different antigen. This opens up a wide range of applications, including redirecting T cells to tumor cells, blocking two different signaling pathways simultaneously, dual targeting of different disease mediators, and delivering payloads to targeted sites.

Multispecific antibodies have various applications. However, in some cases, a multispecific antibody may not have the desired efficacy and it can be difficult to express and purify. There is a need to continue to develop various therapeutics based on multispecific antibodies.

### SUMMARY

This disclosure relates to multispecific antibodies or antigen-binding fragments thereof, wherein the multispecific antibodies or antigen-binding fragments thereof specifically bind to a T cell antigen (e.g., CD3) and/or a tumor-associated antigen (e.g., CEACAM5), or a combination thereof.

In one aspect, the disclosure is related to an antigen-binding protein, comprising (a) an Fc; (b) a first antigen-binding site comprising a VH domain and a VL domain, in some embodiments, the VH domain and the VL domain associate with each other, forming the first antigen-binding site that specifically binds to a T cell antigen; and (c) a second antigen-binding site comprising a single-domain antibody variable domain (VHH) that specifically binds to a tumor-associated antigen, in some embodiments, the first antigen-binding site and the second antigen-binding site are linked to the Fc.

In some embodiments, the first antigen-binding site comprises a single-chain variable fragment (scFv) comprising the VH domain and the VL domain. In some embodiments, the scFv can activate T cells upon binding to the T cell antigen. In some embodiments, the T cell antigen is cluster of differentiation 3 (CD3). In some embodiments, the tumor-associated antigen is cluster of differentiate 20 (CD20), prostate-specific antigen (PSA), prostate stem cell antigen (PSCA), programmed death-ligand 1 (PD-L1), human epidermal growth factor receptor 2 (Her2), human epidermal growth factor receptor 3 (Her3), human epidermal growth factor receptor (Her1), β-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), or carcinoembryonic antigen cell adhesion molecule 5 (CEACAM5), e.g., CEACAM5. In some embodiments, the Fc is human IgG4 Fc.

In some embodiments, the scFv is linked to a CH2 domain in the Fc, optionally via a hinge region. In some embodiments, the hinge region is a human IgG4 hinge region optionally with S228P mutation according to EU numbering.

In some embodiments, the scFv is linked to the C-terminus of a CH3 domain in the Fc. In some embodiments, the scFv is linked to the CH3 domain via a linker peptide.

In some embodiments, the VHH is linked to a CH2 domain in the Fc, optionally via a hinge region. In some embodiments, the hinge region is a human IgG4 hinge region optionally with S228P mutation according to EU numbering.

In some embodiments, the VHH is linked to the C-terminus of a CH3 domain in the Fc. In some embodiments, the VHH is linked to the CH3 domain via a linker peptide.

In some embodiments, the Fc comprises a first polypeptide chain and a second polypeptide chain, in some embodiments, each chain comprises one or more knobs-into-holes mutations.

In one aspect, the disclosure is related to a protein complex, comprising: (a) a first polypeptide comprising from N-terminus to C-terminus: a single-chain variable fragment (scFv), optionally a first hinge region, a first CH2 domain, and a first CH3 domain; and (b) a second polypeptide comprising from N-terminus to C-terminus: a single-domain antibody variable domain (VHH), optionally a second hinge region, a second CH2 domain, and a second CH3 domain, in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 2, in some embodiments, the second polypeptide comprises a sequence that is at least 80%, 90%, 95%, or 100% identical to SEQ ID NO: 11.

In one aspect, the disclosure is related to a protein complex, comprising: (a) a first polypeptide comprising from N-terminus to C-terminus: a single-chain variable fragment (scFv), optionally a first hinge region, a first CH2 domain, a first CH3 domain, optionally a linker peptide, and a single-domain antibody variable domain (VHH); and (b) a second polypeptide comprising from N-terminus to C-terminus: optionally a second hinge region, a second CH2 domain, and a second CH3 domain, in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 6, in some embodiments, the second polypeptide comprises a sequence that is at least 80%, 90%, 95%, or 100% identical to SEQ ID NO: 8.

In one aspect, the disclosure is related to a protein complex, comprising: (a) a first polypeptide comprising from N-terminus to C-terminus: a single-chain variable fragment (scFv), optionally a first hinge region, a first CH2 domain, and a first CH3 domain; and (b) a second polypeptide comprising from N-terminus to C-terminus: optionally a second hinge region, a second CH2 domain, a second CH3 domain, optionally a linker peptide, and a single-domain antibody variable domain (VHH), in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 2, in some embodiments, the second polypeptide comprises a sequence that is at least 80%, 90%, 95%, or 100% identical to SEQ ID NO: 12.

In one aspect, the disclosure is related to a protein complex, comprising: (a) a first polypeptide comprising from N-terminus to C-terminus: a single-domain antibody variable domain (VHH), optionally a first hinge region, a first CH2 domain, and a first CH3 domain; and (b) a second polypeptide comprising from N-terminus to C-terminus: a single-chain variable fragment (scFv), optionally a second hinge region, a second CH2 domain, and a second CH3 domain. In some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 4, in some embodiments, the second polypeptide comprises a sequence that is at least 80%, 90%, 95%, or 100% identical to SEQ ID NO: 9.

In one aspect, the disclosure is related to a protein complex, comprising: (a) a first polypeptide comprising from N-terminus to C-terminus: optionally a first hinge region, a first CH2 domain, a first CH3 domain, optionally a linker peptide, and a single-domain antibody variable domain (VHH); and (b) a second polypeptide comprising from N-terminus to C-terminus: a single-chain variable fragment (scFv), optionally a second hinge region, a second CH2 domain, and a second CH3 domain, in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 5, in some embodiments, the second polypeptide comprises a sequence that is at least 80%, 90%, 95%, or 100% identical to SEQ ID NO: 9.

In one aspect, the disclosure is related to a protein complex, comprising: (a) a first polypeptide comprising from N-terminus to C-terminus: optionally a first hinge region, a first CH2 domain, and a first CH3 domain; and (b) a second polypeptide comprising from N-terminus to C-terminus: a single-chain variable fragment (scFv), optionally a second hinge region, a second CH2 domain, a second CH3 domain, optionally a linker peptide, and a single-domain antibody variable domain (VHH), in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 1, in some embodiments, the second polypeptide comprises a sequence that is at least 80%, 90%, 95%, or 100% identical to SEQ ID NO: 13.

In one aspect, the disclosure is related to a protein complex, comprising: (a) a first polypeptide comprising from N-terminus to C-terminus: a single-domain antibody variable domain (VHH), optionally a first hinge region, a first CH2 domain, a first CH3 domain, optionally a linker peptide, and a single-chain variable fragment (scFv); and (b) a second polypeptide comprising from N-terminus to C-terminus: optionally a second hinge region, a second CH2 domain, and a second CH3 domain, in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 7, in some embodiments, the second polypeptide comprises a sequence that is at least 80%, 90%, 95%, or 100% identical to SEQ ID NO: 8.

In one aspect, the disclosure is related to a protein complex, comprising: (a) a first polypeptide comprising from N-terminus to C-terminus: optionally a first hinge region, a first CH2 domain, a first CH3 domain, optionally a linker peptide, and a single-chain variable fragment (scFv); and (b) a second polypeptide comprising from N-terminus to C-terminus: a single-domain antibody variable domain (VHH), optionally a second hinge region, a second CH2 domain, and a second CH3 domain, in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 3, in some embodiments, the second polypeptide comprises a sequence that is at least 80%, 90%, 95%, or 100% identical to SEQ ID NO: 11.

In one aspect, the disclosure is related to a protein complex, comprising: (a) a first polypeptide comprising from N-terminus to C-terminus: optionally a first hinge region, a first CH2 domain, a first CH3 domain, optionally a first linker peptide, and a single-chain variable fragment (scFv); and (b) a second polypeptide comprising from N-terminus to C-terminus: optionally a second hinge region, a second CH2 domain, a second CH3 domain, optionally a second linker peptide, and a single-domain antibody variable domain (VHH), in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 3, in some embodiments, the second polypeptide comprises a sequence that is at least 80%, 90%, 95%, or 100% identical to SEQ ID NO: 12.

In one aspect, the disclosure is related to a protein complex, comprising: (a) a first polypeptide comprising from N-terminus to C-terminus: a single-domain antibody variable domain (VHH), optionally a first hinge region, a first CH2 domain, and a first CH3 domain; and (b) a second polypeptide comprising from N-terminus to C-terminus: optionally a second hinge region, a second CH2 domain, a second CH3 domain, optionally a linker peptide, and a single-chain variable fragment (scFv), in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 4, in some embodiments, the second polypeptide comprises a sequence that is at least 80%, 90%, 95%, or 100% identical to SEQ ID NO: 10.

In one aspect, the disclosure is related to a protein complex, comprising: (a) a first polypeptide comprising from N-terminus to C-terminus: optionally a first hinge region, a first CH2 domain, and a first CH3 domain; and (b) a second polypeptide comprising from N-terminus to C-terminus: a single-domain antibody variable domain (VHH), optionally a second hinge region, a second CH2 domain, a second CH3 domain, optionally a linker peptide, and a single-chain variable fragment (scFv), in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 1, in some embodiments, the second polypeptide comprises a sequence that is at least 80%, 90%, 95%, or 100% identical to SEQ ID NO: 14.

In one aspect, the disclosure is related to a protein complex, comprising: (a) a first polypeptide comprising from N-terminus to C-terminus: optionally a first hinge region, a first CH2 domain, a first CH3 domain, optionally a first linker peptide, and a single-domain antibody variable domain (VHH); and (b) a second polypeptide comprising from N-terminus to C-terminus: optionally a second hinge region, a second CH2 domain, a second CH3 domain, optionally a second linker peptide, and a single-chain variable fragment (scFv), in some embodiments, the scFv specifically binds to a T cell antigen, in some embodiments, the VHH specifically binds to a tumor-associated antigen. In some embodiments, the T cell antigen is CD3, and the tumor-associated antigen is CEACAM5. In some embodiments, the first polypeptide comprises a sequence that is at least 80%, 90%, 95%, or 100% identical to SEQ ID NO: 5, in some embodiments, the second polypeptide comprises a sequence that is at least 80%, 90%, 95%, or 100% identical to SEQ ID NO: 10.

In some embodiments, the first CH3 domain comprises one or more knob mutations, and the second CH3 domain comprises one or more hole mutations. In some embodiments, the scFv comprises a sequence that is at least 80%, 90%, 95%, or 100% identical to SEQ ID NO: 17. In some embodiments, the VHH comprises a sequence that is at least 80%, 90%, 95%, or 100% identical to SEQ ID NO: 18. In some embodiments, the first hinge region and/or the second hinge region comprise a sequence that is at least 80%, 90%, 95%, or 100% identical to SEQ ID NO: 19. In some embodiments, the first Fc region and/or the second Fc region comprise a sequence that is at least 80%, 90%, 95%, or 100% identical to SEQ ID NO: 20 or 21. In some embodiments, the linker peptide, the first linker peptide, and/or the second linker peptide comprises a sequence that is at least 80%, 90%, 95%, or 100% identical to SEQ ID NO: 15 or one or more repeats (e.g., 1, 2, 3, 4, 5, 6, 7, or 8) of SEQ ID NO: 16.

In one aspect, the disclosure is related to a nucleic acid comprising a polynucleotide encoding the antigen-binding protein as described herein, or the protein complex as described herein. In some embodiments, the nucleic acid is a DNA (e.g., cDNA) or RNA (e.g., mRNA).

In one aspect, the disclosure is related to a vector comprising one or more of the nucleic acids as described herein.

In one aspect, the disclosure is related to a cell comprising the vector as described herein. In some embodiments, the cell is a HEK293F cell or CHO cell.

In one aspect, the disclosure is related to a cell comprising one or more of the nucleic acids as described herein.

In one aspect, the disclosure is related to a method of producing an antibody or an antigen-binding fragment thereof, the method comprising (a) culturing the cell as described herein under conditions sufficient for the cell to produce the antigen-binding protein or protein complex; and (b) collecting the antigen-binding protein or protein complex produced by the cell.

In one aspect, the disclosure is related to an antibody-drug conjugate comprising the antigen-binding protein as described herein, or the protein complex as described herein, covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antigen-binding protein, the protein complex, or the antibody-drug conjugate as described herein, to the subject. In some embodiments, the subject has a cancer expressing CEACAM5. In some embodiments, the cancer is lung cancer, colorectal cancer, head and neck cancer, stomach cancer, pancreatic cancer, urothelial cancer, breast cancer, cervical cancer, or endometrial cancer.

In one aspect, the disclosure is related to a method of decreasing the rate of tumor growth, the method comprising contacting a tumor cell with an effective amount of a composition comprising the antigen-binding protein, the protein complex, or the antibody-drug conjugate as described herein.

In one aspect, the disclosure is related to a method of killing a tumor cell, the method comprising contacting a tumor cell with an effective amount of a composition comprising the antigen-binding protein, the protein complex, or the antibody-drug conjugate as described herein.

In one aspect, the disclosure is related to a pharmaceutical composition comprising the antigen-binding protein as described herein or the protein complex as described herein, and a pharmaceutically acceptable carrier.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multispecific antibodies, bispecific antibodies, single-chain antibodies, diabodies, linear antibodies formed from these antibodies or antibody fragments, and antigen binding protein constructs.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)₂, and Fv fragments, scFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to CEACAM5 may be referred to as a CEACAM5-specific antibody or an anti-CEACAM5 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain antibody. In some embodiments, the VHH is a humanized VHH. In some embodiments, the VHH is a single-domain antibody (sdAb).

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1L show schematic structures of 12 T cell engager (TCB) constructs including a T cell activator (TA1) in the form of scFv and a tumor-associated antigen-targeting moiety (TAA) in the form of sdAb. 4N1 and 4O1 represent the hinge-Fc region of the knob chain (4N1) and the hinge-Fc region of the hole chain (4O1), respectively.

FIGS. 2A-2L show size-exclusion chromatography profiles of 12 TCBs based on a scFv/sdAb combination strategy. The 12 TCBs were purified by a single affinity chromatography step using a Protein A column. Names of the constructs are labeled over each profile. CEA represents an sdAb that binds to CEACAM5, a TAA. sdAbs binding to other TAAs can be used to replace CEA if desired.

FIGS. 3A-3L show images of SDS-PAGE (sodium dodecyl sulphate-polyacrylamide gel electrophoresis) results of 12 TCBs. Each TCB sample was analyzed under either a non-reducing (NR, left of the MW marker) or reducing (R, right of the MW marker) condition. M is the molecular weight (MW) marker. Names of the TCBs are labeled over each gel image. Band sizes are also labeled on the left side of each gel image.

FIGS. 4A-4D show TCB-mediated tumor cell killing curves. FIG. 4A shows efficacy profiles of three TCBs when TA1 was fused to the N-terminus of the knob chain. FIG. 4B shows efficacy profiles of three TCBs when TA1 was fused to the N-terminus of the hole chain. FIG. 4C shows efficacy profiles of three TCBs when TA1 was fused to the C-terminus of the knob chain. FIG. 4D shows efficacy profiles of three TCBs when TA1 was fused to the C-terminus of the hole chain.

FIG. 5 shows amino acid sequences of seven knob chains and seven hole chains.

FIG. 6 lists amino acid sequences discussed in the present disclosure.

### DETAILED DESCRIPTION

This disclosure relates to multispecific antibodies (e.g., bispecific antibodies) or antigen-binding proteins. In one aspect, the multispecific antibodies or antigen-binding proteins can bind to a T cell antigen (e.g., CD3) and/or a tumor-associated antigen (e.g., CEACAM5), or a combination thereof.

In general, bispecific antibodies or multispecific antibodies include two or more antigen-binding sites targeting different antigens or different epitopes of the same antigen. Thus, bispecific antibodies or multispecific antibodies can have more functions than a monospecific antibody. For example, these functions include, but not limited to, stronger binding to an antigen through an avidity effect; co-localization of bound antigens (e.g., Her2 and Her3) on the cell surface and the effect therefrom; increasing the serum half-life of an antibody fragment (e.g., scFv) by linking it to a second antibody fragment that is bound to a protein with a long serum half-life, e.g., albumin or transferrin; and bringing two cells into proximity by binding to an antigen on each of the cells.

Among the purposes of bispecific antibodies or multispecific antibodies, one class of molecules, T cell engagers (TCE), has gained more attention. A TCE is a bispecific antibody or multispecific antibody which binds to an antigen on a T cell and an antigen on another cell simultaneously. CD3 is usually selected as the antigen on the T cell. A cancer or tumor cell is usually selected as the other cell type as discussed above. Through binding to CD3 on T cells and a tumor associated antigen (TAA) on cancer cells, the TCE can induce activation of T cells upon binding to cancer cells and cause the killing of the latter.

Nevertheless, there are some hurdles to overcome in order to generate desirable homogeneous bispecific antibodies or multispecific antibodies. The first hurdle is mismatch of heavy chains that bind to the same target (e.g., antigen or epitope). For example, to generate bispecific antibodies with a desired format, the heavy chains targeting different targets should ideally form a heterodimer. However, the percentage of the desired bispecific or multispecific antibody varies greatly in different constructs. Mutations to induce the formation of knobs-into-holes between two heavy chains can be employed to prevent the formation of homodimers of the heavy chains that bind to the same target. Exemplary amino acid sequences of knob-chain and hole-chain Fc that facilitate heterodimer formation are set forth in SEQ ID NO: 1 and SEQ ID NO: 8, respectively.

The second hurdle to overcome is the mismatch between heavy chain variable regions (VHs) and light chain variable regions (VLs). A monoclonal antibody has two identical Fab fragments, each having a paired VH and VL. By contrast, a bispecific antibody usually has two different heavy chain variable regions and two different light chain variable regions. Therefore, there is a possibility that each VH can bind to the two VLs and each VL can bind to the two VHs. As a result, only half of the formed bispecific antibodies are functional without addressing this mismatch issue.

Several strategies have been designed to disable this mismatch. One solution is to design an antibody with a common light chain. Specifically, a light chain, or more precisely a VL, is selected which can form a dimer with all VHs. This design abrogated the necessity of matching VH and VL with the same target-binding specificity. The shortcoming of this strategy is that the contribution of VL in target binding can be greatly reduced, leading to difficulty finding an optimal VH as the VH will be greatly if not entirely responsible for target binding.

Another solution is the use of CrossMAb technology, in which a VL is fused to heavy chain constant domain 1 (CH1) and becomes part of this heavy chain. Meanwhile, the corresponding VH is fused to the light chain constant region (CL) and becomes a part of this light chain.

The present disclosure provides a different strategy. The reason of employing technologies, e.g., CrossMAb or common light chain, is because conventional antibodies have and need both heavy and light chains to function and/or maintain stability. However, target binding does not necessarily require both heavy and light chains. For example, VH and VL of conventional antibodies can be linked to form a single chain variable fragments (scFv). The variable domain of heavy chain antibodies (VHH), e.g., derived from camelids such as llama, camel, or alpaca, is fully functional for antigen binding. By fusing such antibody fragments (e.g., scFv or VHH) to human Fc with heterodimer preference, bispecific antibodies can be generated without considering the mismatch between heavy and light chains.

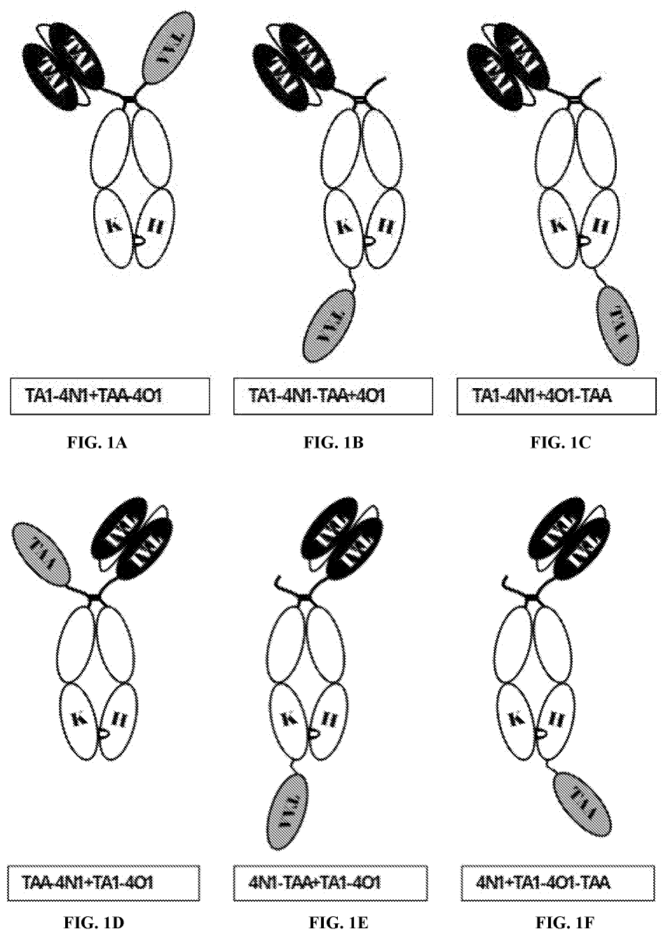

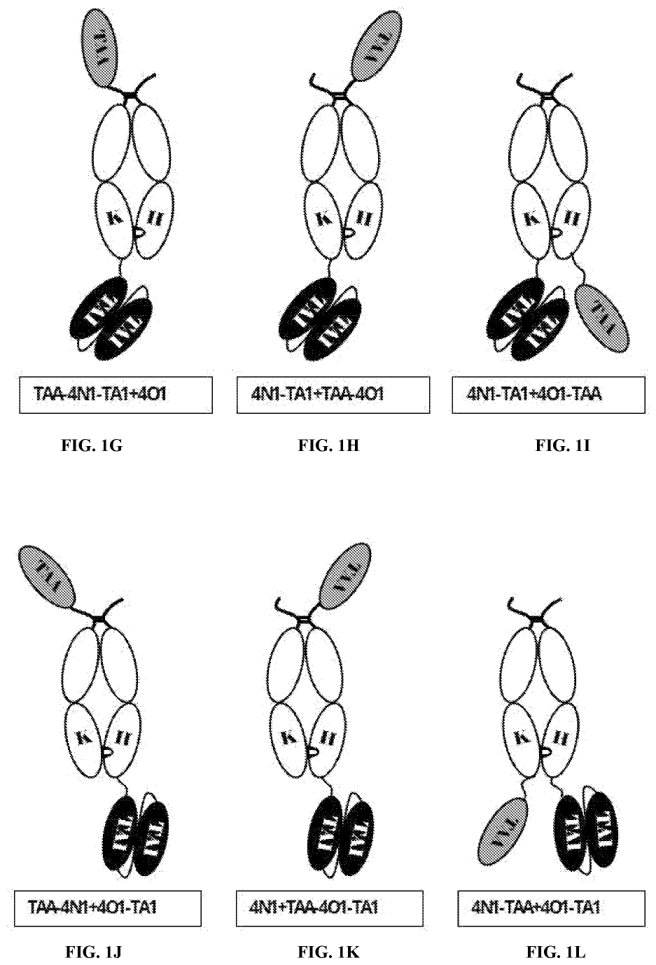

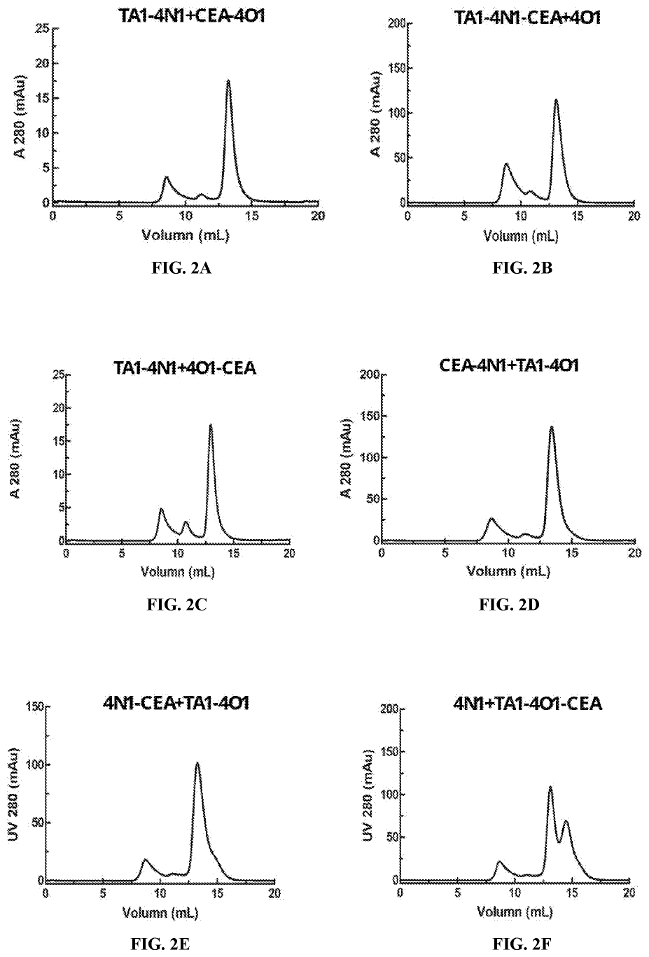

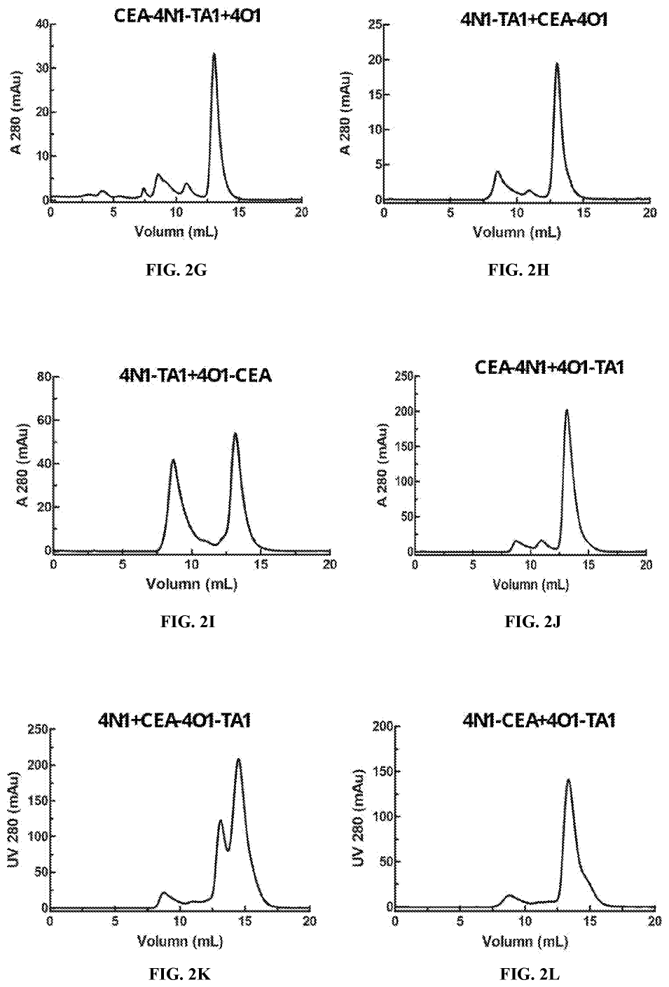

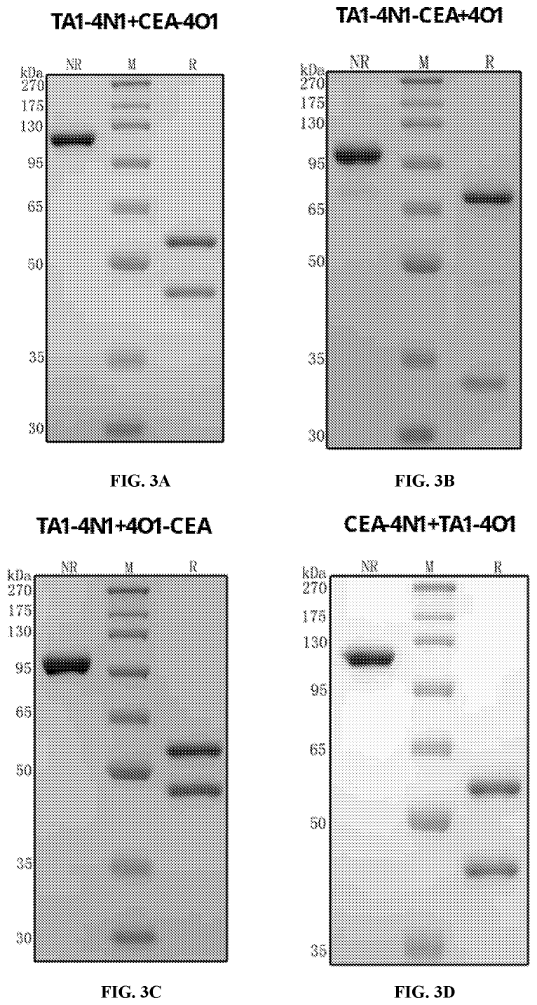

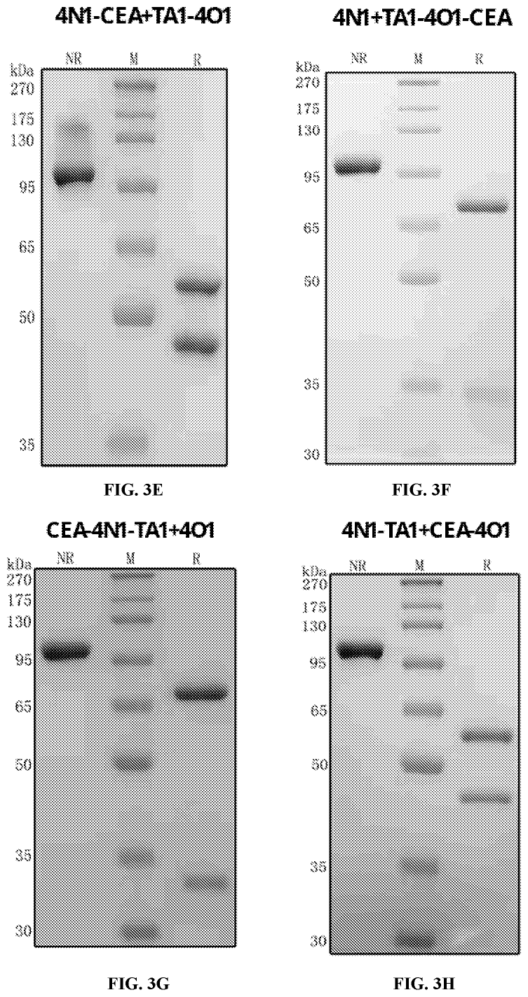

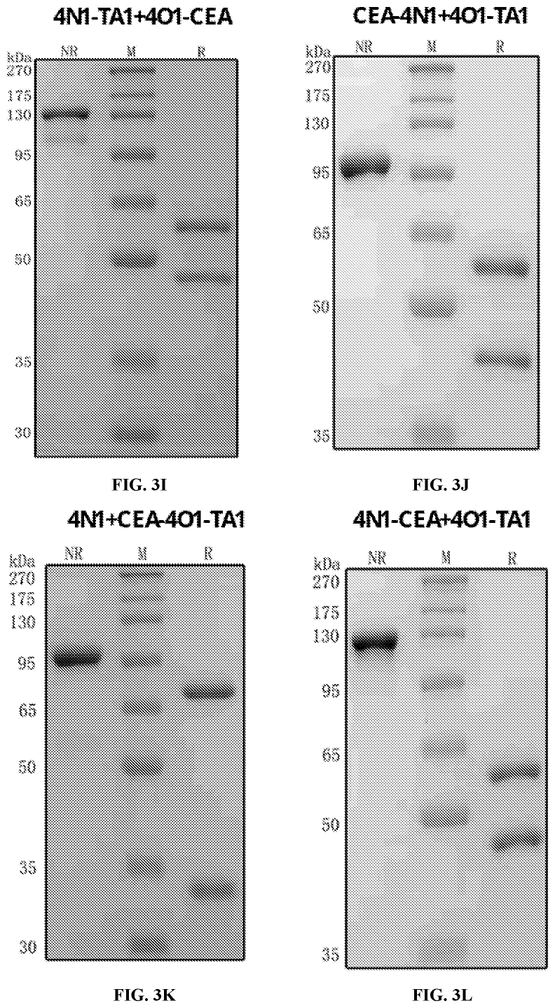

In this disclosure, a scFv, TA1, that can bind to human CD3 and a VHH that can bind to human carcinoembryonic antigen-related cell adhesion molecule 5 (CEACAM5 or CEA) is provided. Either antibody fragment is positioned on each of the four ends of Fc (N- or C-end of knob or hole chain), and the other antibody fragment on the other three available ends of the Fc. Such a design gives rise to 12 different molecules (See FIGS. 1A-1L). The 12 molecules are provided, characterized for their ability to form heterodimer, and tested for their capability in inducing tumor cell killing in the presence of human PBMCs.

In some embodiments, the multispecific antibody or antigen-binding protein can include 1, 2, 3, 4, 5 or more than five single-chain variable fragments (scFv). In some embodiments, the multispecific antibody or antigen-binding protein can include 1, 2, 3, 4, 5, or more than five VHHs. In some embodiments, the scFv can target a T cell antigen (e.g., CD3) or a tumor associated antigen. In some embodiments, the VHH can target a T cell antigen (e.g., CD3) or a tumor associated antigen. The scFv, the VHH, and the multispecific antibody or the antigen binding proteins with various formats are described in detail below.

### Single-Chain Variable Fragment (scFv)

An Fv fragment is an antibody fragment which contains a complete antigen recognition and binding site. This region consists of a dimer of one heavy and one light chain variable domain in tight association, which can be covalent, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding. In some embodiments, the polypeptide linker includes an amino acid sequence that is at least 80%, 90%, 95%, or 100% identical to SEQ ID NO: 24 or one or more repeats (e.g., 1, 2, 3, 4, 5, 6, 7, or 8) of SEQ ID NO: 16.

In some embodiments, the scFv described herein comprises from N-terminus to C-terminus: VH; a polypeptide linker; and VL. In some embodiments, the scFv described herein comprises from N-terminus to C-terminus: VL; a polypeptide linker; and VH. In some embodiments, the linker peptide comprises a sequence that is at least 80%, 85%, 90%, 95%, or 100% identical to SEQ ID NO: 24. In some embodiments, the linker peptide comprises a sequence that is at least or about 80%, 85%, 90%, 95%, or 100% identical to one or more (e.g., 1, 2, 3, 4, 5, 6, 7, or 8) repeats of GGGGS (SEQ ID NO: 16). In some embodiments, the VH comprises a sequence that is an Fv fragment is an antibody fragment which contains a complete antigen recognition and binding site. This region consists of a dimer of one heavy and one light chain variable domain in tight association, for example in scFv.

The disclosure provides e.g., anti-CD3 antibodies, the modified antibodies thereof, the chimeric antibodies thereof, and the humanized antibodies thereof. The disclosure also provides scFv that targets CD3. The scFv can be used in various multispecific antibody constructs as described herein.

The amino acid sequences for various scFv are also provided. In some embodiments, the scFv is at least 80%, 85%, 90%, 91%, 92%, 93%, 94%, 95%, 96%, 97%, 98%, or 99% identical to SEQ ID NO: 17.

In some embodiments, the antibody or an antigen-binding fragment described herein can contain a VH containing VH CDR1 with zero, one or two amino acid insertions, deletions, or substitutions; VH CDR2 with zero, one or two amino acid insertions, deletions, or substitutions; VH CDR3 with zero, one or two amino acid insertions, deletions, or substitutions, and a VL containing one, two, or three of VL CDR1 with zero, one or two amino acid insertions, deletions, or substitutions; VL CDR2 with zero, one or two amino acid insertions, deletions, or substitutions; VL CDR3 with zero, one or two amino acid insertions, deletions, or substitutions, wherein the VH CDRs and VL CDRs are selected from the CDRs of SEQ ID NO: 17.

### Heavy-Chain Antibody Variable Domain (VHH)

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CHI region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

The disclosure provides e.g., anti-CEACAM5 antibodies, the modified antibodies thereof, the chimeric antibodies thereof, and the humanized antibodies thereof. The disclosure also provides VHH of these antibodies. These VHHs can be used in various multispecific antibody constructs as described herein.

The amino acid sequences for various VHH are also provided. In some embodiments, the VHH domain is at least 80%, 85%, 90%, 91%, 92%, 93%, 94%, 95%, 96%, 97%, 98%, or 99% identical to SEQ ID NO: 18.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs of SEQ ID NO: 18.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

The disclosure also provides antibodies or antigen-binding fragments thereof that bind to CEACAM5. The antibodies or antigen-binding fragments thereof contain a heavy-chain antibody variable domain (VHH) comprising or consisting of an amino acid sequence that is at least 80%, 85%, 90%, or 95% identical to SEQ ID NO: 18.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished, e.g., using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy-chain antibody variable domain (VHH).

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

### Structures of Multispecific Antibodies

The multispecific antibodies (e.g., bispecific antibodies) can be designed to include one or more antigen-binding sites that target T cell antigens (e.g., CD3, CD4, and CD8), and include one or more antigen-binding sites that target a tumor-associated antigen. The antigen-binding site can comprise e.g., a Fab, a scFv, a VHH. In some embodiments, the one or more antigen-binding sites that target T cell antigens can comprise a scFv. In some embodiments, the one or more antigen-binding sites that target a tumor-associated antigen can comprise a VHH. The tumor-associated antigen refers to an antigen that is specifically expressed on tumor cell surfaces. These antigens can be used to identify tumor cells. Normal cells rarely express these tumor associated antigens. Some exemplary tumor-associated antigens include, e.g., CD20, PSA, PSCA, PD-L1, Her2, Her3, Her1, β-Catenin, CD19, CEACAM5, EGFR, c-Met, EPCAM, PSMA, CD40, MUC1, and IGF1R, etc.

In some embodiments, a multispecific antibody (e.g., a bispecific antibody) or antigen-binding fragment thereof described herein includes a scFv that specifically binds to a T cell antigen. In some embodiments, the T cell antigen is CD3 (e.g., human CD3). In some embodiments, the T cell antigen is CD28. In some embodiments, the T cell antigen is CD27. In some embodiments, the T cell antigen is CD137. In some embodiments, the T cell antigen is OX40. In some embodiments, the T cell antigen is PD1. In some embodiments, the T cell antigen is CTLA-4. In some embodiments, the T cell antigen is Tim3. In some embodiments, the T cell antigen is LAG-3. In some embodiments, the multispecific antibody or antigen-binding fragment thereof can activate T cells upon binding to the T cell antigen.

In some embodiments, a multispecific antibody (e.g., a bispecific antibody) or antigen-binding fragment thereof described herein includes a VHH that specifically binds to a tumor-associated antigen. In some embodiments, the tumor-associated antigen is CEACAM5 (e.g., human CEACAM5). In some embodiments, the tumor-associated antigen is CEACAM6. In some embodiments, the tumor-associated antigen is EGFR or Her2. In some embodiments, the tumor-associated antigen is EGFR or Her2. In some embodiments, the tumor-associated antigen is Claudin18.2. In some embodiments, the tumor-associated antigen is CD166. In some embodiments, the tumor-associated antigen is Glypican-3. These are just examples of tumor-associated antigens. Listing of them does not mean to limit the utility of only these antigens.

The present disclosure provides antigen-binding protein constructs with various formats as described herein. While not intending to be bound by any theory, it is hypothesized that the in the presence of the target cells (e.g., cancer cells) and T cells, the protein constructs can effectively activate T cells.

In some embodiments, the multispecific antibodies (e.g., bispecific antibodies) are designed to include a scFv that targets CD3. In some embodiments, the multispecific antibodies (e.g., bispecific antibodies) are designed to include a VHH that targets CEACAM5. The multispecific antibodies are described below.

CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex. In some embodiments, the multispecific antibodies target CD3ε.

CEACAM5 (carcinoembryonic antigen-related cell adhesion molecule 5) is a cell surface glycoprotein that represents the founding member of the carcinoembryonic antigen (CEA) family of proteins. It is used as a clinical biomarker for gastrointestinal cancers and may promote tumor development through its role as a cell adhesion molecule. Additionally, CEACAM5 may regulate differentiation, apoptosis, and cell polarity.

The present disclosure provides multispecific antibodies (e.g., bispecific antibodies) that bind to both a T cell antigen and a tumor associated antigen. The multispecific antibodies can be used to treat tumor associated antigen positive cancers in a subject (e.g., a human patient). In some embodiments, the tumor associated antigen positive cancer is CEACAM5-positive (e.g., lung cancer, colorectal cancer, head and neck cancer, stomach cancer, pancreatic cancer, urothelial cancer, breast cancer, cervical cancer, or endometrial cancer).

In general, the multispecific antibody (e.g., bispecific antibody) described herein can be prepared, which includes (a) a first polypeptide including a first Fc region (e.g., CH2 domain and CH3 domain); and (b) a second polypeptide including a second Fc region (e.g., CH2 domain and CH3 domain). In some embodiments, the first Fc region and/or the second Fc region are derived from human IgG4. In some embodiments, the first Fc region includes a sequence that is at least 80%, 85%, 90%, 95%, 96%, 97%, 98%, 99%, or 100% identical to SEQ ID NO: 20. In some embodiments, the second Fc region includes a sequence that is at least 80%, 85%, 90%, 95%, 96%, 97%, 98%, 99%, or 100% identical to SEQ ID NO: 21. In some embodiments, the first Fc region and/or the second Fc region include one or more knobs-into-holes mutations. For example, the first Fc region (e.g., the CH3 domain in the Fc region) can include a tryptophan (Trp) at position 366 according to EU numbering; and the second Fc region (e.g., the CH3 domain in the Fc region) can include one or more of the following a serine (Ser) at position 366, an alanine (Ala) at position 368, and/or a valine (Val) at position 407 according to EU numbering.

In some embodiments, the Fc region is derived from the Fc of any antibody as described herein (e.g., IgG1, IgG2, IgG3, and IgG4). In some embodiments, the Fc region is a human IgG1, IgG2, or IgG4 (e.g., a human IgG4). In some embodiments, the first Fc region and/or the second Fc region include additional mutations relative to the Fc region of a wild-type human IgG (e.g., IgG4). For example, the first Fc region and/or the second Fc region can include a proline (Pro) at position 228 according to EU numbering, to reduce chain exchange of the multispecific antibody. The first Fc region and/or the second Fc region can also include an alanine (Ala) at positions 234 according to EU numbering, to reduce ADCC effect of the multispecific antibody. The first Fc region can include a cysteine (Cys) at position 354 and the second Fc region can further include a cysteine (Cys) at position 349 according to EU numbering, to stabilize the multispecific antibody. The second Fc region can include a lysine (Lys) at position 435 and/or a phenylalanine (Phe) at position 436 according to EU numbering, to reduce binding of the second polypeptide to Protein A. In addition, to improve antibody stability, a glycine (Gly) at position 446 and/or a lysine (Lys) at position 447 of the first Fc region and/or the second Fc region can be deleted. While not intending to be bound by any theory, it is understood by a person skilled in the art that the mutations and deletions described herein can be introduced in either the first Fc region or the second Fc region.

The multispecific antibodies with various structures are described below.

### 1. TA1−4N1+TAA−4O1

As shown in FIG. 1A, a multispecific antibody (e.g., bispecific antibody) can be prepared, which includes (a) a first polypeptide including, preferably from N-terminus to C-terminus: a single-chain variable fragment (scFv), optionally a first hinge region, and a first Fc region (e.g., CH2 domain and CH3 domain); and (b) a second polypeptide including, preferably from N-terminus to C-terminus: a single-domain antibody variable domain (VHH), optionally a second hinge region, and a second Fc region (e.g., CH2 domain and CH3 domain). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 11.

### 2. TA1−4N1−TAA+4O1

As shown in FIG. 1B, a multispecific antibody (e.g., bispecific antibody) can be prepared, which includes (a) a first polypeptide including, preferably from N-terminus to C-terminus: a single-chain variable fragment (scFv), optionally a first hinge region, a first Fc region (e.g., CH2 domain and CH3 domain), optionally a linker peptide, and a single-domain antibody variable domain (VHH); and (b) a second polypeptide including, preferably from N-terminus to C-terminus: optionally a second hinge region, and a second Fc region (e.g., CH2 domain and CH3 domain). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 6. In some embodiments, the second polypeptide comprises a sequence that is at least 80%, 85%, 90%, 95%, 96%, 97%, 98%, 99%, or 100% identical to SEQ ID NO: 8.

### 3. TA1−4N1+4O1+TAA

As shown in FIG. 1C, the multispecific antibody (e.g., bispecific antibody) can be prepared, which includes (a) a first polypeptide including, preferably from N-terminus to C-terminus: a single-chain variable fragment (scFv), optionally a first hinge region, and a first Fc region (e.g., CH2 domain and CH3 domain); and (b) a second polypeptide including, preferably from N-terminus to C-terminus: optionally a second hinge region, a second Fc region (e.g., CH2 domain and CH3 domain), optionally a linker peptide, and a single-domain antibody variable domain (VHH). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 2. In some embodiments, the second polypeptide comprises a sequence that is at least 80%, 85%, 90%, 95%, 96%, 97%, 98%, 99%, or 100% identical to SEQ ID NO: 12.

### 4. TAA−4N1+TA1−4O1

As shown in FIG. 1D, the multispecific antibody (e.g., bispecific antibody) can be prepared, which includes (a) a first polypeptide including, preferably from N-terminus to C-terminus: a single-domain antibody variable domain (VHH), optionally a first hinge region, and a first Fc region (e.g., CH2 domain and CH3 domain); and (b) a second polypeptide including, preferably from N-terminus to C-terminus: a single-chain variable fragment (scFv), optionally a second hinge region, and a second Fc region (e.g., CH2 domain and CH3 domain). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19.

In some embodiments, the first polypeptide comprises a sequence that is at least 80%, 85%, 90%, 95%, 96%, 97%, 98%, 99%, or 100% identical to SEQ ID NO: 4. In some embodiments, the second polypeptide comprises a sequence that is at least 80%, 85%, 90%, 95%, 96%, 97%, 98%, 99%, or 100% identical to SEQ ID NO: 9.

### 5. 4N1−TAA+TA1−4O1

As shown in FIG. 1E, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a linker peptide, and a single-domain antibody variable domain (VHH); and (b) a second polypeptide including, preferably from N-terminus to C-terminus: a single-chain variable fragment (scFv), optionally a second hinge region, and a second Fc region (e.g., CH2 domain and CH3 domain). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 5. In some embodiments, the second polypeptide comprises a sequence that is at least 80%, 85%, 90%, 95%, 96%, 97%, 98%, 99%, or 100% identical to SEQ ID NO: 9.

### 6. 4N1+TA1−4O1−TAA

As shown in FIG. 1F, the multispecific antibody (e.g., bispecific antibody) can be prepared, which includes (a) a first polypeptide including, preferably from N-terminus to C-terminus: optionally a first hinge region, and a first Fc region (e.g., CH2 domain and CH3 domain); and (b) a second polypeptide including, preferably from N-terminus to C-terminus: a single-chain variable fragment (scFv), optionally a second hinge region, a second Fc region (e.g., CH2 domain and CH3 domain), optionally a linker peptide, and a single-domain antibody variable domain (VHH). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 13.

### 7. TAA−4N1−TA1+4O1

As shown in FIG. 1G, the multispecific antibody (e.g., bispecific antibody) can be prepared, which includes (a) a first polypeptide including, preferably from N-terminus to C-terminus: a single-domain antibody variable domain (VHH), optionally a first hinge region, a first Fc region (e.g., CH2 domain and CH3 domain), optionally a linker peptide, and a single-chain variable fragment (scFv); and (b) a second polypeptide including, preferably from N-terminus to C-terminus: optionally a second hinge region, and a second Fc region (e.g., CH2 domain and CH3 domain). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 7. In some embodiments, the second polypeptide comprises a sequence that is at least 80%, 85%, 90%, 95%, 96%, 97%, 98%, 99%, or 100% identical to SEQ ID NO: 8.

### 8. 4N1−TA1+TAA−4O1

As shown in FIG. 1H, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a linker peptide, and a single-chain variable fragment (scFv); and (b) a second polypeptide including, preferably from N-terminus to C-terminus: a single-domain antibody variable domain (VHH), optionally a second hinge region, and a second Fc region (e.g., CH2 domain and CH3 domain). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 3. In some embodiments, the second polypeptide comprises a sequence that is at least 80%, 85%, 90%, 95%, 96%, 97%, 98%, 99%, or 100% identical to SEQ ID NO: 11.

### 9. 4N1−TA1+4O1−TAA

As shown in FIG. 1I, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a first linker peptide, and a single-chain variable fragment (scFv); and (b) a second polypeptide including, preferably from N-terminus to C-terminus: optionally a second hinge region, a second Fc region (e.g., CH2 domain and CH3 domain), optionally a second linker peptide, and a single-domain antibody variable domain (VHH). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 3. In some embodiments, the second polypeptide comprises a sequence that is at least 80%, 85%, 90%, 95%, 96%, 97%, 98%, 99%, or 100% identical to SEQ ID NO: 12.

### 10. TAA−4N1+4O1−TA1

As shown in FIG. 1J, the multispecific antibody (e.g., bispecific antibody) can be prepared, which includes (a) a first polypeptide including, preferably from N-terminus to C-terminus: a single-domain antibody variable domain (VHH), optionally a first hinge region, and a first Fc region (e.g., CH2 domain and CH3 domain); and (b) a second polypeptide including, preferably from N-terminus to C-terminus: optionally a second hinge region, a second Fc region (e.g., CH2 domain and CH3 domain), optionally a linker peptide, and a single-chain variable fragment (scFv). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 3. In some embodiments, the second polypeptide comprises a sequence that is at least 80%, 85%, 90%, 95%, 96%, 97%, 98%, 99%, or 100% identical to SEQ ID NO: 12.

### 11. 4N1+TAA−4O1−TA1

As shown in FIG. 1K, the multispecific antibody (e.g., bispecific antibody) can be prepared, which includes (a) a first polypeptide including, preferably from N-terminus to C-terminus: optionally a first hinge region, and a first Fc region (e.g., CH2 domain and CH3 domain); and (b) a second polypeptide including, preferably from N-terminus to C-terminus: a single-domain antibody variable domain (VHH), optionally a second hinge region, a second Fc region (e.g., CH2 domain and CH3 domain), optionally a linker peptide, and a single-chain variable fragment (scFv). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the linker peptide includes a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 14.

### 12. 4N1−TAA+4O1−TA1

As shown in FIG. 1L, the multispecific antibody (e.g., bispecific antibody) can be prepared, which includes (a) a first polypeptide including, preferably from N-terminus to C-terminus: optionally a first hinge region, a first Fc region (e.g., CH2 domain and CH3 domain), optionally a first linker peptide, and a single-domain antibody variable domain (VHH); and (b) a second polypeptide including, preferably from N-terminus to C-terminus: optionally a second hinge region, a second Fc region (e.g., CH2 domain and CH3 domain), optionally a second linker peptide, and a single-chain variable fragment (scFv). In some embodiments, the scFv specifically binds to a T cell antigen (e.g., CD3). In some embodiments, the VHH specifically binds to a tumor-associated antigen (e.g., CEACAM5).

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 20. In some embodiments, the second polypeptide comprises a sequence that is at least 80%, 85%, 90%, 95%, 96%, 97%, 98%, 99%, or 100% identical to SEQ ID NO: 21.

In some embodiments, the scFv can target CD3 (e.g., human CD3), and comprises a sequence that is at least 80%, 85%, 90%, 95%, 96%, 97%, 98%, 99%, or 100% identical to SEQ ID NO: 17. In some embodiments, the VHH can target CEACAM5, and comprises a sequence that is at least 80%, 85%, 90%, 95%, 96%, 97%, 98%, 99%, or 100% identical to SEQ ID NO: 18. In some embodiments, the first hinge region and/or the second hinge region are derived from the hinge region of human IgG4. In some embodiments, the first hinge region and/or the second hinge region include a sequence that is at least 80%, 85%, 90%, 95%, 96%, 97%, 98%, 99%, or 100% identical to SEQ ID NO: 19. In some embodiments, the first and/or the second linker peptide include a sequence that is at least 80%, 85%, 90%, 95%, 96%, 97%, 98%, 99%, or 100% identical to SEQ ID NO: 15, or one or more repeats (e.g., 1, 2, 3, 4, 5, 6, 7, or 8) of SEQ ID NO: 16.

In some embodiments, the first polypeptide comprises a sequence that is at least 80%, 85%, 90%, 95%, 96%, 97%, 98%, 99%, or 100% identical to SEQ ID NO: 5. In some embodiments, the second polypeptide comprises a sequence that is at least 80%, 85%, 90%, 95%, 96%, 97%, 98%, 99%, or 100% identical to SEQ ID NO: 10.

### Antigen-Binding Protein Construct Characteristics

The anti-CD3, anti-CEACAM5, or anti-CD3/CEACAM5 antigen-binding protein construct (e.g., antibodies, bispecific antibodies, trispecific antibodies, multispecific antibodies, or antibody fragments thereof) can include an antigen binding site that is derived from any anti-CD3 antibody, anti-CEACAM5 antibody, or any antigen-binding fragment thereof as described herein.

In some embodiments, the antibodies or antigen-binding fragments thereof described herein are CEACAM5 antagonist. In some embodiments, the antibodies or antigen-binding fragments thereof are CEACAM5 agonist. In some embodiments, the antibodies or antigen-binding fragments thereof as described herein are CD3 antagonist. In some embodiments, the antibodies or antigen-binding fragments thereof are CD3 agonist.

In some embodiments, the antibodies, or antigen-binding fragments thereof described herein can bind to CD3 and/or CEACAM5, thereby bridging T cells and target cells; activating T cells; and inducing directly killing the cancer cells by the T cells.

In some embodiments, the antibody (or antigen-binding fragments thereof) specifically binds to a T cell antigen (e.g., CD3) or tumor-associated antigen (e.g., CEACAM5) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁻⁴ M, less than 1×10⁻⁵ M, less than 1×10⁻⁶ M, less than 1×10⁻⁷ M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, Kd is greater than 1×10⁻⁴ M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from Kd by the formula Ka=1/Kd.

General techniques for measuring the affinity of an antibody for an antigen include, e.g., ELISA, RIA, and surface plasmon resonance (SPR).

In some embodiments, the expression level of the antibodies, the antigen binding fragments thereof, or the antigen-binding protein constructs described herein is at least or about 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 50, 100, 200, 300, 400, 500, 600, 700, 800, 900, 1000, 2000, 3000, 4000, 5000, 6000, 7000, 8000, 9000, 10000, 20000, 50000, or 100000 mg/L, as determined using the method described herein. In some embodiments, the percentage of multispecific antibody (e.g., bispecific antibody) formed, as determined by size-exclusion chromatography as described herein, is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 93%, at least 94%, at least 95%, at least 96%, or at least 97% of the total protein level.

In some embodiments, the antibodies, the antigen binding fragments thereof, or the antigen-binding protein constructs described herein have a cell killing EC50 of less than or about 10 pM, less than or about 9 pM, less than or about 8 pM, less than or about 7 pM, less than or about 6 pM, less than or about 5 pM, less than or about 4 pM, less than or about 3 pM, less than or about 2 pM, less than or about 1 pM, less than or about 0.5 pM, as determined using the method described herein. In some embodiments, the antibodies or antigen binding fragments thereof, or the antigen-binding protein constructs described herein have a cell killing EC50 value that is about 0.1 pM to about 10 pM, about 1 pM to 10 pM, about 5 pM to 10 pM, about 0.1 pM to 5 pM, or about 0.1 pM to 1 pM. In some embodiments, the antibodies, the antigen binding fragments thereof, or the antigen-binding protein constructs described herein have a cell killing EC50 value that is less than or about 50%, less than or about 30%, less than or about 20%, less than or about 10%, less than or about 5%, less than or about 1% as compared to that of an isotype control antibody.

In some embodiments, the antibodies, the antigen binding fragments thereof, or the antigen-binding protein constructs have a functional Fc region. In some embodiments, effector function of a functional Fc region is antibody-dependent cell-mediated cytotoxicity (ADCC) and/or complement-dependent cytotoxicity (CDC). In some embodiments, the Fc region is human IgG1, human IgG2, human IgG3, or human IgG4.

In some embodiments, the antibodies, the antigen binding fragments thereof, or the antigen-binding protein constructs do not have a functional Fc region. For example, the antibodies or antigen binding fragments are Fab, Fab′, F(ab′)₂, and Fv fragments. In some embodiments, the antibodies or antigen binding fragments, or the antigen-binding protein constructs have a Fc region that includes one or more mutations to reduce the effector function. In some embodiments, the antibodies, the antigen binding fragments thereof, or the antigen-binding protein constructs described herein do not have antibody-dependent cell-mediated cytotoxicity (ADCC) or complement dependent cytotoxicity (CDC).

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the bispecific antibody described herein (e.g., a CD3/CEACAM5 bispecific antibody) has an asymmetric structure comprising: 2, 3, 4, 5, or 6 antigen binding sites. In some embodiments, the multispecific antibody described herein comprises 2, 3, 4, 5, or 6 antigen binding sites (e.g., antigen binding Fab domains, scFV, or nanobody (VHH)) that target a tumor-associated antigen (e.g., CEACAM5). In some embodiments, the tumor-associated antigen (e.g., CEACAM5) binding Fab domain comprises the same variable domain sequence. In some embodiments, the tumor-associated antigen (e.g., CEACAM5) binding Fab domain comprises different variable domain sequences.

### Antibodies and Antigen Binding Fragments

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region. Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3): 9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Kontermann, R., & Dübel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody or antigen-binding protein can include an intact immunoglobulin molecule (e.g., IgG1, IgG2a, IgG2b, IgG3, IgM, IgD, IgE, IgA) or fragments thereof. The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody or antigen-binding protein can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen-binding domain” or “antigen-binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFv has two heavy chain variable domains, and two light chain variable domains. In some embodiments, the scFv has two antigen binding regions, and the two antigen binding regions can bind to the respective target antigens with different affinities.

In some embodiments, the antibodies or antigen-binding fragments thereof can bind to two different antigens or two different epitopes. In some embodiments, the antibodies or antigen-binding fragments thereof can bind to three different antigens or three different epitopes.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

In some embodiments, the Fc region can be further modified to increase or decrease effector functions as well as serum half-life.

Any of the antibodies, antigen-binding fragments thereof, or antigen-binding proteins described herein can be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of the antibodies, antigen-binding fragments thereof, or antigen-binding proteins in vitro (e.g., in tissue culture or when stored as a pharmaceutical composition) or in vivo (e.g., in a human).

In some embodiments, the antibodies, antigen-binding fragments thereof, or antigen-binding proteins (e.g., multispecific antibodies) described herein can be conjugated to a therapeutic agent. The antibody-drug conjugate comprising the antibodies, antigen-binding fragments thereof, or antigen-binding proteins can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multispecific antibody or antigen-binding fragment thereof described herein (e.g., a CD3/CEACAM5 multispecific antibody) binds to a T cell antigen (e.g., CD3) with a binding affinity that is about 50%, about 60%, about 70%, about 80%, about 90%, about 100%, about 110%, about 120%, about 130%, about 140%, about 150%, or about 200% to that of an antibody (e.g., an anti-CD3 antibody) comprising the same antigen binding region (e.g., Fab, scFv or VHH) of the multi-specific antibody.

In some embodiments, the multispecific antibody or antigen-binding fragment thereof described herein (e.g., a CD3/CEACAM5 multispecific antibody) binds to a tumor-associated antigen (e.g., CEACAM5) with a binding affinity that is about 50%, about 60%, about 70%, about 80%, about 90%, about 100%, about 110%, about 120%, about 130%, about 140%, about 150%, or about 200% to that of an antibody (e.g., an anti-CEACAM5 antibody) comprising the same antigen binding region (e.g., Fab, scFv or VHH) of the multi-specific antibody.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof or the antigen-binding protein constructs by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HEK293 cells; and plant cells. Appropriate culture mediums and conditions for the host cells described herein are known in the art.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies or Antigen-Binding Protein Constructs

An isolated fragment of human protein (e.g., CD3 or CEACAM5) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Variants of the antibodies, antigen-binding fragments described herein, or the antigen-binding protein constructs can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions T366W. The other heavy chain can have one or more the following substitutions T366S, L368A, and Y407V. Furthermore, a substitution (-ppcpScp-->-ppcpPcp-) can also be introduced at the hinge regions of both substituted IgG.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

### Methods of Treatment

The methods described herein include methods for the treatment of disorders associated with cancer. Generally, the methods include administering a therapeutically effective amount of engineered multispecific antibodies (e.g., bispecific antibodies) or the antigen-binding protein constructs as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., antigen-binding protein constructs) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, antigen-binding protein constructs, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing CEACAM5.

In some embodiments, the cancers are lung cancers, colorectal cancer, head and neck cancer, stomach cancer, pancreatic cancer, urothelial cancer, breast cancer, cervical cancer, or endometrial cancer.

In some embodiments, the cancer cells described herein is cell lines, e.g., LS-174T cells. In some embodiments, the cancer cells have an elevated CEACAM5 level, e.g., at least 10%, at least 20%, at least 30%, at least 40%, at least 50% higher than non-cancerous cells.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antigen-binding protein constructs, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, an antigen-binding protein construct, or an antibody-drug conjugate is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the agent used.

Effective amounts and schedules for administering the antibodies, antigen-binding protein constructs, antibody-encoding polynucleotides, antibody-drug conjugates, and/or compositions disclosed herein may be determined empirically, and making such determinations is within the skill in the art.

A typical dosage of an effective amount of an antibody or antigen-binding protein construct is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antigen-binding protein constructs, antibody-drug conjugates, or pharmaceutical composition (e.g., any of the antibodies, antigen-binding fragments, antigen-binding protein constructs, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody, antigen-binding fragment thereof, or the antigen-binding protein construct can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antigen-binding protein constructs, antigen binding proteins, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies, antigen-binding fragments thereof, or antigen-binding protein constructs (e.g., any of the antibodies, antibody fragments, or antigen-binding protein constructs described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies, antigen-binding fragments, or antigen-binding protein constructs described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, antigen-binding protein constructs, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody, antibody fragment, or antigen-binding protein constructs in vivo.

The pharmaceutical compositions can be included in a container, pack, or dispenser together with instructions for administration. The disclosure also provides methods of manufacturing the antibodies, antigen binding fragments thereof, or antigen-binding protein constructs for various uses as described herein.

### EXAMPLES

The invention is further described in the following examples, which do not limit the scope of the invention described in the claims.

### Materials and Methods

### Cell Culture and Reagents

Tumor cell line LS-174T was acquired from the Chinese Academy of Sciences in Shanghai, China (Cell Bank, Cat #TCHu 32). LS-174T cells were cultured in Dulbecco's modified Eagle's medium (DMEM) (Gibco, Life Technologies, China) supplemented with 10% heat-inactivated (HI) fetal bovine serum (Gibco, Life Technologies, USA) and 1% Penicillin/Streptomycin (Hyclone). Fresh human peripheral blood mononuclear cells (PBMCs) were purchased from Leide Bioscience Co., Ltd., Guangzhou, China. PBMCs were maintained in RPMI-1640 medium (Gibco, Life Technologies, China) supplemented with 10% HI fetal bovine serum (Gibco, Life Technologies, USA) and 1% Penicillin/Streptomycin (Hyclone). Cells were stained for viability determination with trypan blue and counted using an Automated Cell Counter (IC1000, Countstar, China).

### Molecular Cloning

The IgG4 constant regions (e.g., the hinge-Fc regions) was generated by site-specific mutagenesis from a wild-type human IgG4 format using molecular biology techniques.

Generation of bispecific antibodies was achieved by fusing a T cell activator (TA1), and a tumor-associated antigen-targeting moiety (TAA), to various locations of the generated IgG4 constant regions using molecular biology techniques. The TA1 can be a CD3-binding scFv (single-chain variable fragment) as described herein. In some cases, an sdAb that binds to carcinoembryonic antigen-related cell adhesion molecule 5 (CEACAM5), designated CEA, was used as the TAA. Sequences of all clones were verified before entering antibody production phase.

### Antibody Production

All generated bispecific antibodies (bsAbs) were produced in a transient expression system using HEK293F as the expression host. Briefly, 0.3×10⁶ 293F cells were used to inoculate 300 mL KOP293 medium (Zhuhai Kairui, Zhuhai, China) and allowed for cell proliferation for 2-4 days with 120 rpm in 5% CO₂. Next, the cells were used to inoculate 100 mL of KPM Transfection Medium (Zhuhai Kairui) to reach a cell density of 1×10⁶ cells/mL, and allowed for growth for overnight. 100 μg plasmid DNA encoding each pair of constructed knob and hole chains were mixed with 600 μg PEI by slowly adding the PEI solution to the plasmid solution. After incubation at room temperature (RT) for 15 minutes, the plasmid-PEI mixture was added to the 293F cells, and the cells were cultured as described above. The cells were allowed for growth for 8 days. 2 mL KT-feed (50×, Zhuhai Kairui) was added after 24 hours of growth. Harvest of culture supernatant was conducted by centrifugation and filtration through a 0.22 μm filter. Antibodies were purified by Protein A affinity chromatography using MabSelect™ PrismA FF column (GE) following manufacturer's instructions.

### PBMC Co-Cultivation Assays

A total of 5×10³ cells in RPMI1640 medium (supplemented with 10% FBS and 2 mM glutamine) of the target cell line (LS-174T) were seeded in 96-well plates (Day 0). A dilution series of respective antibodies was performed in assay media and added to target cells. On Day 0, a total of 5×10⁴ PBMCs were added to each well, and the final reaction volume was adjusted to 200 μl. Percentage viability of target cell was measured by lactate dehydrogenase (LDH) release of live cells. LDH was measured after 72 hours using the Cell Counting Kit-8 (CK04, Dojindo, Japan) according to the manufacturer's recommendations. The results were analyzed as mean and standard deviation (SD) from triplicate wells and plotted as 4-parameter non-linear regression fittings using GraphPad Prism 9 software (GraphPad Software, San Diego, CA, USA).

To assess the bispecific-antibody-mediated activation of lymphocytes, PBMCs were isolated from blood of healthy human donors and co-cultivated with respective antibody dilution and target cells. Representative killing curve of each bispecific antibody was generated accordingly.

### Example 1. Design of TCBs Based on scFv and sdAb Combination

For the construction of TCBs, or more specifically “T-cell-engaging bsAbs,” IgG4 Fc was selected for its low capability in inducing antibody-dependent cytotoxicity (ADCC) and complement-dependent cytotoxicity (CDC). Because the purpose of the study was to create appropriate scFv/sdAb combinations for the production of TCBs, mutations enabling bispecific antibody formation as well as other mutations were also introduced in the IgG4 Fc, as shown in the table below.

TABLE 1
Mutations in constant regions of the TCBs

| Chains | Mutations | Functions |
|---|---|---|
| Knob | S228P | Reduction of chain exchange |
| | F234A, L235A | Reduction of ADCC |
| | S354C | Antibody stabilization |
| | T366W | Knob mutation |
| | Del446G, Del447K | Improved stability |
| Hole | S228P | Reduction of chain exchange |
| | F234A, L235A | Reduction of ADCC |
| | S349C | Antibody stabilization |
| | T366S, L368A, Y407V | Hole mutations |
| | H435R, Y436F | Reduced binding of hole chain |
| | | to Protein A |
| | Del446G, Del447K | Improved stability |

Note:
Amino acid positions are based on EU numbering.

To exploit full potentials of scFv/sdAb combination for the generation of TCBs with Fc, all possible combinations were tested by fusing both a CD3-binding scFv (shown as “TA1”) and an sdAb that binds to CEACAM5 (shown as “TAA” or “CEA”) to two of the four ends (e.g., N-terminus and C-terminus of the knob chain; N-terminus and C-terminus of the hole chain) of the Fc. This strategy generated 12 different TCB molecules. Schematic structures of the 12 TCBs are shown in FIGS. 1A-1L, respectively.

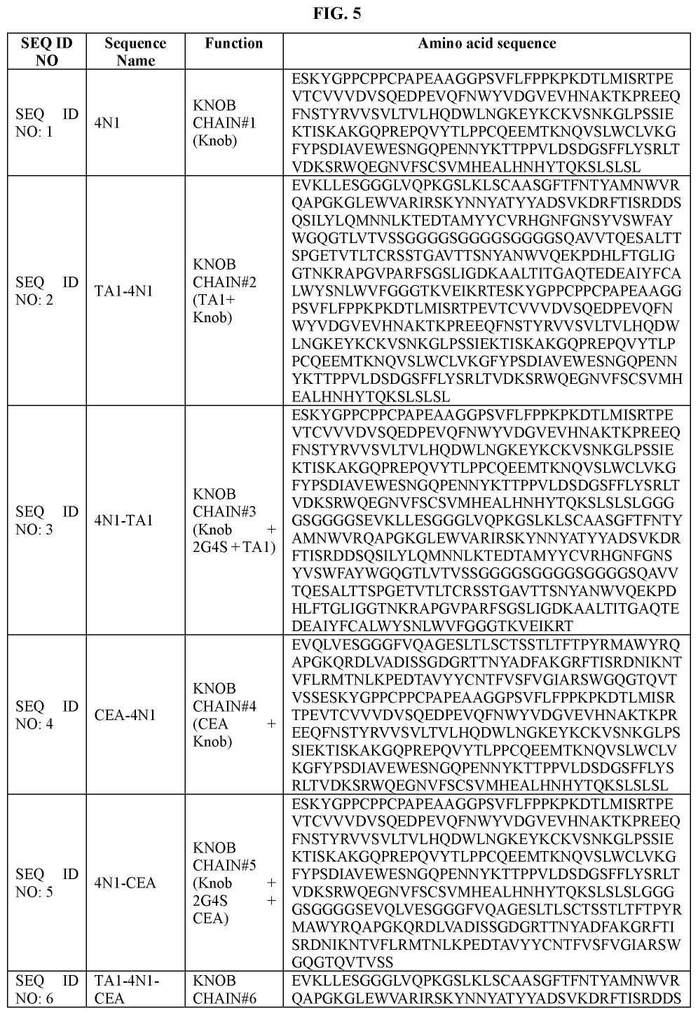

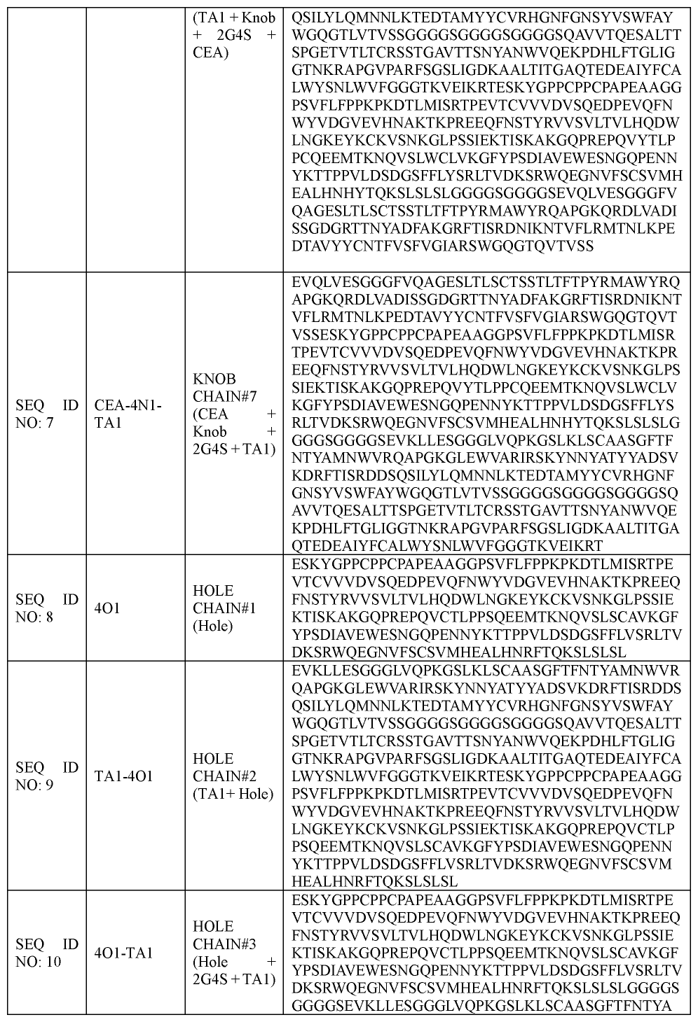

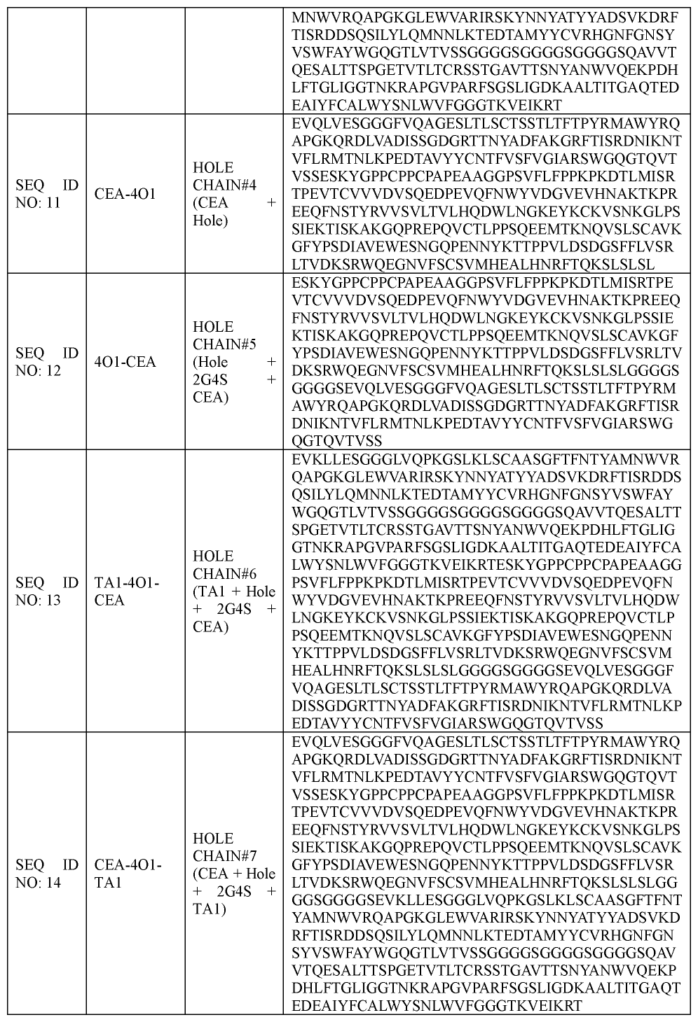

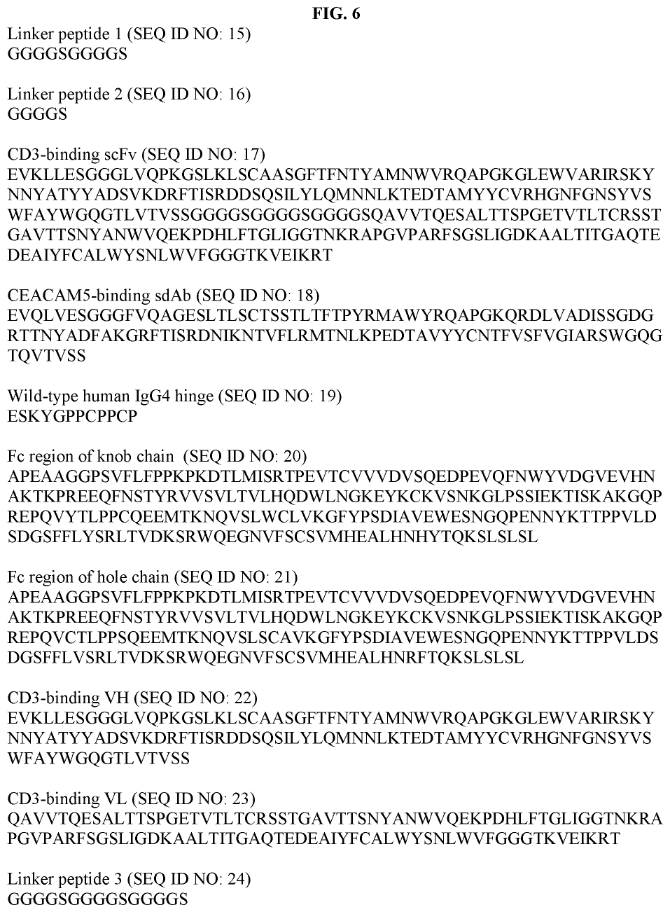

Generation of the 12 TCBs, or bsAbs, involved in construction of a total of seven knob chains and seven hole chains. Sequences of the seven knob chains and the seven hole chains are listed in FIG. 5.

### Example 2. Production of TCBs

The Fc region of IgG4 from the 12 TCBs are the same, each having mutations to enable the formation of knobs-into-holes and additional mutations for other functions. The amino acid sequence of the hinge-Fc region of the knob chain (N) is set forth in SEQ ID NO: 1, and the amino acid sequence of the hinge-Fc region of the hole chain (O) is set forth in SEQ ID NO: 8. Wild-type human IgG4 hinge (SEQ ID NO: 19) was used when connecting a scFv or sdAb to the N-terminus of either the knob or the hole chain. A linker peptide sequence GGGGSGGGGS (SEQ ID NO: 15) was used to connect the scFv or sdAb to the C-terminus of either the knob or the hole chain. Nucleic acid sequences encoding all 12 TCB molecules were constructed using molecular biology techniques. The 12 TCB molecules were produced using a transient expression system in 293F cells. The produced antibodies were purified by affinity chromatography using a Protein A column.

The expression was repeated three times and similar results were obtained. One set of representative results is shown in the table below. Both expression levels and percentage of bsAbs varied largely, ranging from 4 mg/L to 27 mg/L for expression and 26% to 87% for percentage of bsAbs in a transient expression setting.

TABLE 2
Expression levels, percentage of formed
bsAbs, and EC₅₀ of the TCBs

| | | Expression | Percentage | |
|---|---|---|---|---|
| | FIG. | level | of bsAb | EC₅₀ |
| TCBs | No. | (mg/L) | (%) | (pM) |
| TA1 − 4N1 + CEA − 4O1 | 1A, 4A | 8 | 79 | 3.1 |
| TA1 − 4N1 − CEA + 4O1 | 1B, 4A | 4 | 51 | * |
| TA1 − 4N1 + 4O1 − CEA | 1C, 4A | 9 | 79 | * |
| CEA − 4N1 + TA1 − 4O1 | 1D, 4B | 6 | 77 | 0.005 |
| 4N1 − CEA + TA1 − 4O1 | 1E, 4B | 13 | 72 | * |
| 4N1 + TA1 − 4O1 − CEA | 1F, 4B | 16 | 46 | * |
| CEA − 4N1 − TA1 + 4O1 | 1G, 4C | 7 | 63 | 0.4 |
| 4N1 − TA1 + CEA − 4O1 | 1H, 4C | 8 | 71 | 6.0 |
| 4N1 − TA1 + 4O1 − CEA | 1I, 4C | 12 | 73 | 6.7 |
| CEA − 4N1 + 4O1 − TA1 | 1J, 4D | 11 | 87 | 9.0 |
| 4N1 + CEA − 4O1 − TA1 | 1K, 4D | 27 | 26 | * |
| 4N1 − CEA + 4O1 − TA1 | 1L, 4D | 24 | 76 | * |

Note:
“*” represents molecules with no clear transitional phase in its efficacy profile.

Six of the TCB molecules had expression levels above 10 mg/L, and the other six had expression levels below 10 mg/L. It is noteworthy that five of the six TCB molecules with above 10 mg/L expression levels had the TA1 fused to the hole chain (See FIGS. 1D-1F, 1J-1L, and Table 2). Among the six TCB molecules in which the TA1 was fused to the hole chain, even higher expression was observed among those when the TA1 was fused to the C-terminus (See FIGS. 1J-1L, and Table 2). The two clones that exhibited the highest expression levels (i.e., 4N1+CEA−4O1−TA1 and 4N1−CEA+4O1−TA1; See FIGS. 1K and 1L) are among this group. This result suggests that fusion of TA1 to the hole chain, especially to the C-terminus of the hole chain, is favorable for higher TCB expression.

In addition, four of the twelve TCB molecules had lower than 70% percentage of formed bsAbs, ranging from 26% for 4N1+CEA−4O1−TA1 (See FIG. 1K) to 63% for CEA−4N1−TA1+4O1 (See FIG. 1G). The other eight TCB molecules had above 70% percentage of formed bsAbs, ranging from 71% for 4N1−TA1+CEA−4O1 (See FIG. 1H) to 87% for CEA−4N1+4O1−TA1 (See FIG. 1J). All four TCB molecules with lower than 70% percentage of formed bsAbs had both the T cell activating unit (e.g., TA1) and target-engaging unit (e.g., TAA or CEA) on the same chain, either knob or hole, and all eight TCB molecules with above 70% percentage of formed bsAbs had the two units fused to two different chains. It is therefore recommended to arrange a scFv (e.g., TA1) and an sdAb (e.g., TAA or CEA) on different chains when generating Fc-based T-cell-engaging bsAbs using the scFv/sdAb combination strategy.

All generated antibodies were further purified to have more than 95% homogeneity by cation exchange chromatography. The antibodies were used to assess the capability of inducing T-cell-dependent and target-dependent tumor cell killings.

### Example 3. Efficacy of Generated TCBs

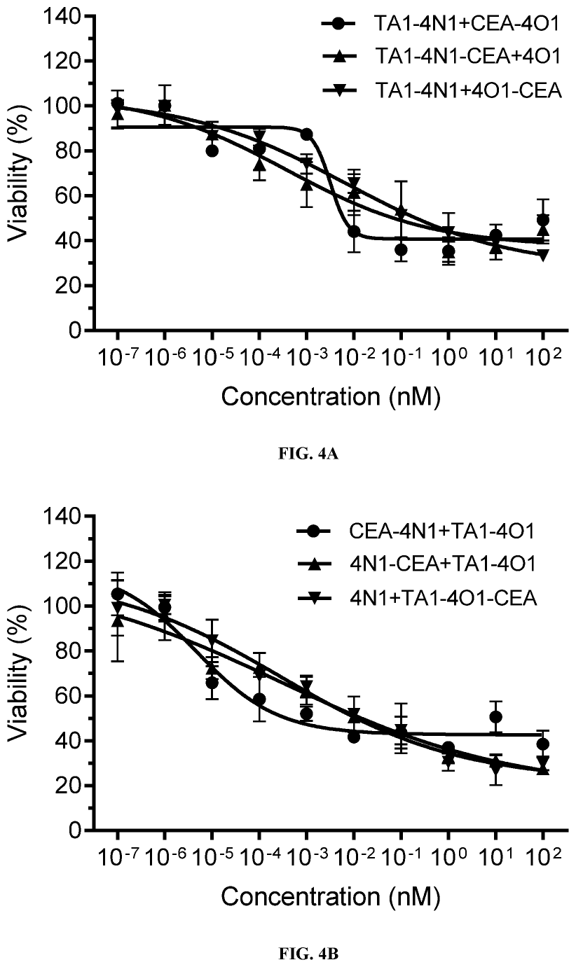

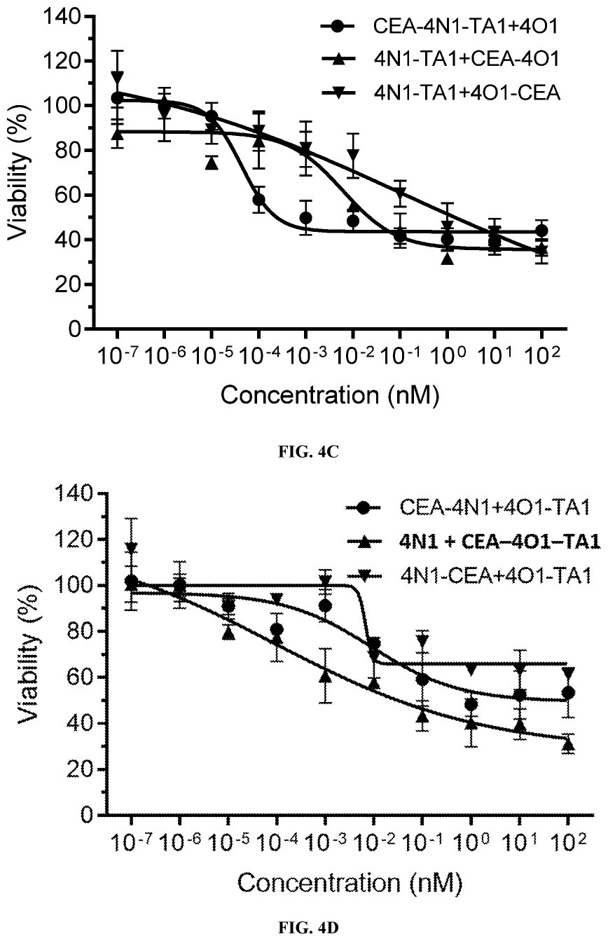

To assess bispecific-antibody-mediated activation of lymphocytes, PBMCs were isolated from blood of healthy human donors and co-cultivated for 48 hours with respective antibody dilution and target cells. Percentage viability of the target cells was measured by LDH release of live cells. Results were expressed as mean and SD from triplicate wells, then plotted as 4-parameter non-linear regression fitting using Graphpad Prism software. A representative killing curve of each bispecific antibody is shown in FIGS. 4A-4D.

Although all twelve bispecific antibodies included identical T cell activation unit, TA1, and target binding unit, CEA, they exhibited largely different tumor cell killing profiles.

The first observation was that no clear transitional phase was observed in six of the twelve molecules (i.e., TA1−4N1−CEA+4O1 and TA1−4N1+4O1−CEA in FIG. 4A; 4N1−CEA+TA1−4O1 and 4N1+TA1−4O1−CEA in FIG. 4B; 4N1+CEA−4O1−TA1 and 4N1−CEA+4O1−TA1 in FIG. 4D), and hence no EC₅₀ was calculated for them (See Table 2). Given the high purity of all tested molecules and clear transitional phases observed in other molecules, the results suggest that the lack of a clear transitional phase was due to some intrinsic characteristics of the molecules.

High quality bioassay data are essential in both antibody platform establishment and screening for good individual antibodies. Poor assay results can be caused by factors such as poor intrinsic protein characterization, low protein purity, poor protein-expressing cell conditions, contaminations, etc. As clear transitional phases were observed from other molecules in the same experiment, the unclear transitional phases of some molecules can be caused by their intrinsic characteristics. Such molecules are not recommended in the selection of an antibody platform including a scFv and an sdAb, as they have unfavorable efficacy profiles.

In particular, three of the four molecules with the CD3-binding scFv (TA1) and CEACAM5-binding sdAb (TAA or CEA) on the same chain with characteristics of low percentage of formed bispecific antibodies (See Table 2) are among the six unfavorable molecules.

As shown in FIGS. 4A and 4C, clear transitional phases were observed on four molecules. EC₅₀ of the four molecules were determined as 3.1 pM for TA1−4N1+CEA−4O1, 0.4 pM for CEA−4N1−TA1+4O1, 6.0 pM for 4N1−TA1+CEA−4O1, and 6.7 pM for 4N1−CEA+4O1−TA1 (See Table 2). The single digit picomolar or sub-picomolar range efficacy of the molecules indicate that all these molecules are very potent TCBs. As shown in FIGS. 4B and 4D, transitional phases were also observed on the other two molecules CEA−4N1+TA1−4O1 and CEA−4N1+4O1−TA1, with a calculated EC₅₀ of 0.005 PM and 9.0 pM, respectively. However, their transitions were slow in comparison to the other four molecules.

Regarding the scFv and sdAb arrangement on the Fc, no common structural features were observed among the four TCBs having clear and sharp transitional phases. Specifically, TA1−4N1+CEA−4O1 has both units at the N-terminus (See FIG. 1A); 4N1−TA1+4O1−CEA has both units at the C-terminus (See FIG. 1I); CEA−4N1−TA1+4O1 has both units on the knob chain (i.e., CEACAM5-binding sdAb at the N-terminus and CD3-binding scFv at the C-terminus; See FIG. 1G); and 4N1−TA1+CEA−4O1 has the two units on two different chains and at different terminus (See FIG. 1H). These results clearly demonstrated that, the arrangement of the CD3-binding scFv and the target-binding sdAb can have significant impact on the efficacy.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antigen-binding protein, comprising (a) an Fc;
(b) a first antigen-binding site comprising a VH domain and a VL domain, wherein the VH domain and the VL domain associate with each other, forming the first antigen-binding site that specifically binds to a T cell antigen; and
(c) a second antigen-binding site comprising a single-domain antibody variable domain (VHH) that specifically binds to a tumor-associated antigen,
wherein the first antigen-binding site and the second antigen-binding site are linked to the Fc.

2. The antigen-binding protein of claim 1, wherein the first antigen-binding site comprises a single-chain variable fragment (scFv) comprising the VH domain and the VL domain.

3. The antigen-binding protein of claim 2, wherein the scFv can activate T cells upon binding to the T cell antigen.

4. The antigen-binding protein of any one of claims 1-3, wherein the T cell antigen is cluster of differentiation 3 (CD3).

5. The antigen-binding protein of any one of claims 1-4, wherein the tumor-associated antigen is cluster of differentiate 20 (CD20), prostate-specific antigen (PSA), prostate stem cell antigen (PSCA), programmed death-ligand 1 (PD-L1), human epidermal growth factor receptor 2 (Her2), human epidermal growth factor receptor 3 (Her3), human epidermal growth factor receptor (Her1), B-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), or carcinoembryonic antigen cell adhesion molecule 5 (CEACAM5), e.g., CEACAM5.

6. The antigen-binding protein of any one of claims 1-5, wherein the Fc is human IgG4 Fc.

7. The antigen-binding protein of any one of claims 2-6, wherein the scFv is linked to a CH2 domain in the Fc, optionally via a hinge region.

8. The antigen-binding protein of claim 7, wherein the hinge region is a human IgG4 hinge region optionally with S228P mutation according to EU numbering.

9. The antigen-binding protein of any one of claims 2-6, wherein the scFv is linked to the C-terminus of a CH3 domain in the Fc.

10. The antigen-binding protein of claim 9, wherein the scFv is linked to the CH3 domain via a linker peptide.

11. The antigen-binding protein of any one of claims 1-10, wherein the VHH is linked to a CH2 domain in the Fc, optionally via a hinge region.

12. The antigen-binding protein of claim 11, wherein the hinge region is a human IgG4 hinge region optionally with S228P mutation according to EU numbering.

13. The antigen-binding protein of any one of claims 1-10, wherein the VHH is linked to the C-terminus of a CH3 domain in the Fc.

14. The antigen-binding protein of claim 13, wherein the VHH is linked to the CH3 domain via a linker peptide.

15. The antigen-binding protein of any one of claims 1-14, wherein the Fc comprises a first polypeptide chain and a second polypeptide chain, wherein each chain comprises one or more knobs-into-holes mutations.

16. A protein complex, comprising:
(a) a first polypeptide comprising from N-terminus to C-terminus: a single-chain variable fragment (scFv), optionally a first hinge region, a first CH2 domain, and a first CH3 domain; and
(b) a second polypeptide comprising from N-terminus to C-terminus: a single-domain antibody variable domain (VHH), optionally a second hinge region, a second CH2 domain, and a second CH3 domain,
wherein the scFv specifically binds to a T cell antigen, wherein the VHH specifically binds to a tumor-associated antigen.

17. The protein complex of claim 16, wherein the T cell antigen is CD3, and the tumor-associated antigen is CEACAM5.

18. The protein complex of claim 16 or 17, wherein the first polypeptide comprises a sequence that is at least 80%, 90%, 95%, or 100% identical to SEQ ID NO: 2, wherein the second polypeptide comprises a sequence that is at least 80%, 90%, 95%, or 100% identical to SEQ ID NO: 11.

19. A protein complex, comprising:
(a) a first polypeptide comprising from N-terminus to C-terminus: a single-domain antibody variable domain (VHH), optionally a first hinge region, a first CH2 domain, and a first CH3 domain; and
(b) a second polypeptide comprising from N-terminus to C-terminus: a single-chain variable fragment (scFv), optionally a second hinge region, a second CH2 domain, and a second CH3 domain,
wherein the scFv specifically binds to a T cell antigen, wherein the VHH specifically binds to a tumor-associated antigen.

20. The protein complex of claim 19, wherein the T cell antigen is CD3, and the tumor-associated antigen is CEACAM5.

21. The protein complex of claim 19 or 20, wherein the first polypeptide comprises a sequence that is at least 80%, 90%, 95%, or 100% identical to SEQ ID NO: 4, wherein the second polypeptide comprises a sequence that is at least 80%, 90%, 95%, or 100% identical to SEQ ID NO: 9.

22. A protein complex, comprising:
(a) a first polypeptide comprising from N-terminus to C-terminus: a single-domain antibody variable domain (VHH), optionally a first hinge region, a first CH2 domain, a first CH3 domain, optionally a linker peptide, and a single-chain variable fragment (scFv); and
(b) a second polypeptide comprising from N-terminus to C-terminus: optionally a second hinge region, a second CH2 domain, and a second CH3 domain,
wherein the scFv specifically binds to a T cell antigen, wherein the VHH specifically binds to a tumor-associated antigen.

23. The protein complex of claim 22, wherein the T cell antigen is CD3, and the tumor-associated antigen is CEACAM5.

24. The protein complex of claim 22 or 23, wherein the first polypeptide comprises a sequence that is at least 80%, 90%, 95%, or 100% identical to SEQ ID NO: 7, wherein the second polypeptide comprises a sequence that is at least 80%, 90%, 95%, or 100% identical to SEQ ID NO: 8.

25. A protein complex, comprising:
(a) a first polypeptide comprising from N-terminus to C-terminus: optionally a first hinge region, a first CH2 domain, a first CH3 domain, optionally a linker peptide, and a single-chain variable fragment (scFv); and
(b) a second polypeptide comprising from N-terminus to C-terminus: a single-domain antibody variable domain (VHH), optionally a second hinge region, a second CH2 domain, and a second CH3 domain,
wherein the scFv specifically binds to a T cell antigen, wherein the VHH specifically binds to a tumor-associated antigen.

26. The protein complex of claim 25, wherein the T cell antigen is CD3, and the tumor-associated antigen is CEACAM5.

27. The protein complex of claim 25 or 26, wherein the first polypeptide comprises a sequence that is at least 80%, 90%, 95%, or 100% identical to SEQ ID NO: 3, wherein the second polypeptide comprises a sequence that is at least 80%, 90%, 95%, or 100% identical to SEQ ID NO: 11.

28. A protein complex, comprising:
(a) a first polypeptide comprising from N-terminus to C-terminus: a single-domain antibody variable domain (VHH), optionally a first hinge region, a first CH2 domain, and a first CH3 domain; and
(b) a second polypeptide comprising from N-terminus to C-terminus: optionally a second hinge region, a second CH2 domain, a second CH3 domain, optionally a linker peptide, and a single-chain variable fragment (scFv),
wherein the scFv specifically binds to a T cell antigen, wherein the VHH specifically binds to a tumor-associated antigen.

29. The protein complex of claim 28, wherein the T cell antigen is CD3, and the tumor-associated antigen is CEACAM5.

30. The protein complex of claim 28 or 29, wherein the first polypeptide comprises a sequence that is at least 80%, 90%, 95%, or 100% identical to SEQ ID NO: 4, wherein the second polypeptide comprises a sequence that is at least 80%, 90%, 95%, or 100% identical to SEQ ID NO: 10.

31. A protein complex, comprising:
(a) a first polypeptide comprising from N-terminus to C-terminus: optionally a first hinge region, a first CH2 domain, a first CH3 domain, optionally a first linker peptide, and a single-domain antibody variable domain (VHH); and
(b) a second polypeptide comprising from N-terminus to C-terminus: optionally a second hinge region, a second CH2 domain, a second CH3 domain, optionally a second linker peptide, and a single-chain variable fragment (scFv),
wherein the scFv specifically binds to a T cell antigen, wherein the VHH specifically binds to a tumor-associated antigen.

32. The protein complex of claim 31, wherein the T cell antigen is CD3, and the tumor-associated antigen is CEACAM5.

33. The protein complex of claim 31 or 32, wherein the first polypeptide comprises a sequence that is at least 80%, 90%, 95%, or 100% identical to SEQ ID NO: 5, wherein the second polypeptide comprises a sequence that is at least 80%, 90%, 95%, or 100% identical to SEQ ID NO: 10.

34. The protein complex of any one of claims 16-33, wherein the first CH3 domain comprises one or more knob mutations, and the second CH3 domain comprises one or more hole mutations.

35. The protein complex of any one of claims 16-34, wherein the scFv comprises a sequence that is at least 80%, 90%, 95%, or 100% identical to SEQ ID NO: 17.

36. The protein complex of any one of claims 16-35, wherein the VHH comprises a sequence that is at least 80%, 90%, 95%, or 100% identical to SEQ ID NO: 18.

37. The protein complex of any one of claims 16-36, wherein the first hinge region and/or the second hinge region comprise a sequence that is at least 80%, 90%, 95%, or 100% identical to SEQ ID NO: 19.

38. The protein complex of any one of claims 16-37, wherein the first Fc region and/or the second Fc region comprise a sequence that is at least 80%, 90%, 95%, or 100% identical to SEQ ID NO: 20 or 21.

39. The protein complex of any one of claims 22-38, wherein the linker peptide, the first linker peptide, and/or the second linker peptide comprises a sequence that is at least 80%, 90%, 95%, or 100% identical to SEQ ID NO: 15 or one or more repeats (e.g., 1, 2, 3, 4, 5, 6, 7, or 8) of SEQ ID NO: 16.

40. A nucleic acid comprising a polynucleotide encoding the antigen-binding protein of any one of claims 1-15, or the protein complex of any one of claims 16-39.

41. The nucleic acid of claim 40, wherein the nucleic acid is a DNA (e.g., cDNA) or RNA (e.g., mRNA).

42. A vector comprising one or more of the nucleic acids of claim 40 or 41.

43. A cell comprising the vector of claim 42.

44. The cell of claim 43, wherein the cell is a HEK293F cell or CHO cell.

45. A cell comprising one or more of the nucleic acids of claim 43 or 44.

46. A method of producing an antibody or an antigen-binding fragment thereof, the method comprising
(a) culturing the cell of any one of claims 43-45 under conditions sufficient for the cell to produce the antigen-binding protein or protein complex; and
(b) collecting the antigen-binding protein or protein complex produced by the cell.

47. An antibody-drug conjugate comprising the antigen-binding protein of any one of claims 1-15, or the protein complex of any one of claims 16-39, covalently bound to a therapeutic agent.

48. The antibody drug conjugate of claim 47, wherein the therapeutic agent is a cytotoxic or cytostatic agent.

49. A method of treating a subject having cancer, the method comprising administering a therapeutically effective amount of a composition comprising the antigen-binding protein of any one of claims 1-15, the protein complex of any one of claims 16-39, or the antibody-drug conjugate of claim 47 or 48, to the subject.

50. The method of claim 49, wherein the subject has a cancer expressing CEACAM5.

51. The method of claim 49 or 50, wherein the cancer is lung cancer, colorectal cancer, head and neck cancer, stomach cancer, pancreatic cancer, urothelial cancer, breast cancer, cervical cancer, or endometrial cancer.

52. A method of decreasing the rate of tumor growth, the method comprising contacting a tumor cell with an effective amount of a composition comprising the antigen-binding protein of any one of claims 1-15, the protein complex of any one of claims 16-39, or the antibody-drug conjugate of claim 47 or 48.

53. A method of killing a tumor cell, the method comprising
contacting a tumor cell with an effective amount of a composition comprising the antigen-binding protein of any one of claims 1-15, the protein complex of any one of claims 16-39, or the antibody-drug conjugate of claim 47 or 48.

54. A method of killing a tumor cell, the method comprising
contacting a tumor cell with an effective amount of a composition comprising the antigen-binding protein of any one of claims 1-15, the protein complex of any one of claims 16-39, or the antibody-drug conjugate of claim 47 or 48.

55. A pharmaceutical composition comprising the antigen-binding protein of any one of claims 1-15, or the protein complex of any one of claims 16-39, and a pharmaceutically acceptable carrier.
